(19) United States
(12) Patent Application Publication
(10) Pub. No.: US 2015/0182428 A1
(43) Pub. Date: Jul. 2, 2015

(54) ANTIPERSPIRANT AND DEODORANT COMPOSITIONS AND METHODS FOR USING THE SAME

(71) Applicant: The Dial Corporation, Scottsdale, AZ (US)

(72) Inventor: Catherine Schmit, Glendale, AZ (US)

(21) Appl. No.: 14/144,733

(22) Filed: Dec. 31, 2013

(51) Int. Cl. A61K 8/28; A61K 8/67; A61K 8/97; A61Q 15/00; A61K 8/27; A61K 8/26

(52) U.S. Cl. CPC A61K 8/28; A61K 8/27; A61K 8/26; A61K 8/97; A61Q 15/00; A61K 8/678

(57) ABSTRACT

Antiperspirant and deodorant compositions and methods for using the same are provided. In one exemplary embodiment, an antiperspirant and deodorant composition includes an antiperspirant active compound based upon an astringent metallic salt, a skin lubricating agent that reduces a coefficient of friction between skin and a razor blade applied to the skin, and an anti-inflammatory agent that reduces effects of skin irritation and damage.

### TECHNICAL FIELD

The present disclosure generally relates to antiperspirant and deodorant compositions. More particularly, the present disclosure relates to antiperspirant and deodorant compositions with lubricating agents and anti-inflammatory agents and methods for preparing the same.

### BACKGROUND

Antiperspirant and deodorant compositions are popular personal care products used to prevent or eliminate perspiration and body odor caused by perspiration. Antiperspirant products, including for example sticks, aerosols, gels, emulsions, and roll-on antiperspirants are desired by a large majority of the population because of the presence of active antiperspirant compounds that minimize or prevent the secretion of perspiration by blocking or plugging ducts of sweat-secreting glands, such as those located at the underarms. Antiperspirants typically include an active antiperspirant compound in a carrier that permits the antiperspirant product to be applied to the skin by swiping or rubbing the stick across the skin, typically of the underarm. Upon application, the carrier coats the skin or evaporates, releasing the active antiperspirant compound from the antiperspirant product upon exposure to moisture to form plugs in the sweat ducts.

Active antiperspirant compounds reduce underarm wetness and odor by migrating into openings of the sweat gland ducts and reacting with proteins therein to form antiperspirant plugs, which mechanically prevent sweat from escaping the ducts. Two types of sweat glands are present in the underarm region. The first type of sweat gland, apocrine sweat glands, terminate and secrete at the top of hair follicles. As such, active antiperspirant compounds should migrate into the hair follicle to access the apocrine sweat gland duct and block secretion. The second type of sweat gland, eccrine sweat glands, open directly onto the skin. Eccrine sweat is responsible for the largest volume of sweat that causes underarm wetness. As with apocrine glands, active antiperspirant compounds migrate into the eccrine gland openings and form plugs, which reduce underarm wetness.

It is commonly known in the art that many consumers who apply antiperspirant and deodorant products shave their underarm region, sometimes several times per week, or even daily. It is further appreciated that shaving may cause skin irritation, particularly in the underarm region. Various other products exist in the marketplace that contain materials to help sooth or minimize irritation of the underarm skin, which are designed for use those consumers that shave their underarms. The shaving process can irritate and damage the underarm skin, and it is for that reason that these products are occasionally formulated with materials such as aloe, allantoin, and panthenoic acid and related materials, to minimize or heal such skin irritation and skin damage.

In addition to these irritation-minimizing products, many shaving devices, such as razors and the like, include a strip of lubricating material, which is provided to reduce the damage to the skin, especially in the underarm region, that shaving may cause. Lubricating materials are known to reduce the coefficient of friction between the skin and the area to be shaved, such as the underarm region, thus reducing the potential for damage to the skin.

Thus, at any given time, a consumer may use a razor with a lubricating strip integrated therewith, along with or followed by an anti-irritation product, followed by an antiperspirant and deodorant product. The need to purchase and use a plurality of products for personal care of the underarm region on a daily basis may be undesirable for the consumer.

Accordingly, it is desirable to provide antiperspirant and deodorant compositions that reduce the incidence of underarm damage during shaving of the underarm region. Additionally, it is desirable to provide antiperspirant and deodorant compositions that reduce inflammation that may be caused by shaving the underarm region. Furthermore, other desirable features and characteristics of the present disclosure will become apparent from the subsequent detailed description the appended claims, taken in conjunction with the accompanying drawings and background.

### BRIEF SUMMARY

Antiperspirant and deodorant compositions and methods for using the same are provided. In one exemplary embodiment, an antiperspirant and deodorant composition includes an antiperspirant active compound based upon an astringent metallic salt, a skin lubricating agent that reduces a coefficient of friction between skin and a razor blade applied to the skin, and an anti-inflammatory agent that reduces effects of skin irritation and damage.

In another exemplary embodiment, a method for using an antiperspirant composition includes applying to the underarm region an antiperspirant composition including an antiperspirant active compound based upon an astringent metallic salt, a skin lubricating agent that reduces a coefficient of friction between skin and a razor blade applied to the skin, and an anti-inflammatory agent that reduces effects of skin irritation and damage, and subsequent to applying the antiperspirant composition, shaving the underarm region using a razor.

This brief summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### DETAILED DESCRIPTION

The following detailed description is merely exemplary in nature and is not intended to limit the disclosed embodiments or the application and uses of the disclosed embodiments. Furthermore, there is no intention to be bound by any theory presented in the preceding background or the following detailed description.

The various embodiments contemplated herein relate to personal care antiperspirant and deodorant products that exhibit lubricating and anti-inflammatory properties and methods for using the antiperspirant and deodorant compositions. In an exemplary embodiment, the antiperspirant and deodorant product includes at least a lubricating compound and an anti-inflammatory compound. Exemplary (non-limiting) lubricating compounds and exemplary anti-inflammatory compounds are provided in greater detail below in the paragraphs that follow. The lubricating agents and exemplary anti-inflammatory agents may be provided in an antiperspirant and deodorant product that further includes antiperspirant active ingredients, fragrances, emollients, antioxidants, and the like. The antiperspirant and deodorant compositions may be provided in accordance with a plurality of formulations. For example, these formulations may include, but are not limited to, aerosols, pumps, sprays, liquids, roll-ons, lotions, creams, sticks, and soft solids, among others. Exemplary formulations for stick antiperspirant and deodorants and aerosol antiperspirant and deodorants are provided in greater detail below. Still further, methods for using the described deodorant compositions are provided in greater detail below.

### Skin Lubricating Compounds

In one embodiment, the antiperspirant and deodorant composition further includes one or more skin lubricating compounds. The skin lubricating compound serves to reduce the aggressiveness and increase the ease with which the user can shave, and further to reduce skin abrasion during shaving, thereby minimizing the damage to the underarm skin that may occur during shaving. Exemplary skin lubricating compounds include slip modifiers, which are compounds used to reduce the friction between the surface of the razor blade used for shaving and the skin (such as the underarm skin) to which the razor is applied, but do not react chemically with the compounds to which they are added. Numerous slip modifying compounds are known in the art, and include quaternary ammonium compounds (including salts), synthetic polymers (including salts), glycosaminoglycans, fatty esters, hydrocarbons, i.e., oils and waxes, silicone polymers, siloxanes and silanes, soaps, and fatty esters. The skin lubricating compound may be added to the antiperspirant composition in an amount between about 1-20% by weight, about 1-10% by weight, or about 1-5% by weight of the total antiperspirant composition. An exemplary, non-limiting listing of skin lubricating compounds thereof is set forth in Table 1, below.

TABLE 1

| | |
|---|---|
| Acrylamide/Ethalkonium Chloride Acrylate Copolymer | Dipentaerythrityl Tetrabehenate/Polyhydroxystearate |
| Acrylamide/Ethyltrimonium Chloride Acrylate | Diphenyl Dimethicone Crosspolymer |
| Acrylic Acid/VP Crosspolymer | Ditrimethylolpropane Tetra Palm Kemelate |
| Acrylonitrile/Methyl Methacrylate Copolymer | Glyceryl Diethylsiloxanes |
| Adipic Acid/Neopentyl Glycol Crosspolymer | Glyceryl Dimethylsiloxanes |
| Algae Exopolysaccharides | Glycol Dimethacrylate Crosspolymer |
| Behenyl Olivate | Gossypium Herbaceum (Cotton) Powder |
| Bis-Aminopropyl Dimethicone/IPDI Copolymer | Hexafluoropropylene/Tetrafluoroethylene Copolymer |
| Bis-Hydroxyethoxypropyl Dimethicone Beeswax Esters | Hibiscus Cannabinus Stem Powder |
| Bis-Hydroxyethoxypropyl Dimethicone Isostearate | Hydrogenated Camellia Oleifera Seed Oil |
| Bis-Vinyl Dimethicone/Dimethicone Copolymer | Hydrogenated Evening Primrose Oil |
| Bis-Vinyldimethicone/PEG-10 Dimethicone | Hydrogenated Grapeseed Oil |
| Crosspolymer | Hydrogenated Hazelnut Oil |
| Bletilla Striata Root Powder | Hydrogenated Hemp Seed Oil |
| Boron Nitride | Hydrogenated Ostrich Oil |
| Brassica Alcohol | Hydrogenated Sesame Seed Oil |
| Butene/Propylene Copolymer | Hydroxycyclohexyl Ethyltrisiloxane Sapucainhate |
| Butylethylpropanediol Dimer Dilinoleate | Kaolin |
| C4-24 Alkyl Dimethicone/Divinyldimethicone | Lauryl Methacrylate/Sodium Methacrylate Crosspolymer |
| Crosspolymer | Lauryl Phenylisopropyl Methicone |
| Cannabis Sativa Stem Powder | Lauryl Trimethicone |
| Cellulose | Lithium/Potassium/Iron/Titanium Oxides |
| Cetrimonium Dimethicone PEG-7 Phthalate | Lithium/Potassium/Titanium Oxides |
| Chalk | Lithium Stearate |
| Coco/Sunfloweramidopropyl Betaine | Magnesium Aluminum Silicate |
| C24-30 Olefin | Magnesium Cocoate |
| C28-36 Olefin | Magnesium Myristate |
| Dibutyl Fumarate/Elexadecene/Tetradecene Copolymer | Magnesium Palmitate |
| Dibutyl Maleate/Hexadecene/Tetradecene Copolymer | Polyperfluoroethoxymethoxy Difluoroethyl PEG Ether |
| Diisoamyl Malate | Polyurethane-16 |
| Myristyl Trisiloxane | Polyvinylidene Difluoride |
| Nephrite Powder | Potassium Behenoyl Hydrolyzed Rice Protein |
| Octyldodecyl Olivate | PPG-3 Benzyl Ether Ethylhexanoate |
| PEG-6 Dimethicone | PPG-3 Benzyl Ether Myristate |
| PEG-4 Distearyl Ether | PTFE |
| PEG-200 Hydrogenated Castor Oil/IPDI Copolymer | Pueraria Lobata Starch |
| PEG/PPG-12/16 Dimethicone | Raffinose Isostearate |
| PEG/PPG-12/18 Dimethicone | Saccharomyces/Opal/Mother ofPearl/Turquoise |
| PEG/PPG-16/8 Dimethicone | Shea Butter Glycerides |
| PEG/PPG-20/23 Dimethicone | Silica Dimethyl Silylate |
| PEG/PPG-23/6 Dimethicone | Silk Powder |
| Perfluorocyclohexylmethanol | Sodium/Aluminum Hydroxide/Oxalate/Sulfate |
| Perfluoroheptane | Sodium Stearyl Fumarate |
| Perfluoromethylcyclohexane | Stearyl Trimethicone |
| Perfluoromethyldecalin | Synthetic Sapphire |
| Perfluorononyl Dimethicone | Talc |
| Perfluorononylethyl Dimethicone/Methicone Copolymer | Titanium Nitride |
| Phaseolus Radiatus Seed Powder | Tri-PPG-3 Myristyl Ether Citrate |
| Phenylisopropyl Dimethicone | Undecylcrylene Dimethicone |
| Pisum Sativum (Pea) Starch | Zinc Laurate |
| Polydiethylsiloxane | Zinc Myristate |
| Polyester-11 | Zinc Palmitate |
| Polyethyloxazoline | Zinc Rosinate |
| Polyglyceryl-3 Behenate | Zinc Stearate |
| Polyglyceryl-6 Behenate | |
| Polyperfluoroethoxymethoxy Difluoroethyl PEG | |

### Anti-Inflammatory Compounds

In one embodiment, the antiperspirant and deodorant composition further includes one or more anti-inflammatory compounds. The anti-inflammatory compound serves to reduce the effects of skin irritation and skin damage caused by shaving. For example, skin irritation is understood to be any change in the skin that in humans triggers sensorial discomfort or/and is marked by a dry, reddened and/or inflamed appearance of the skin. The term sensorial discomfort naturally also includes conditions such as itching or pain. Exemplary skin anti-inflammatory compounds may include, for example, active anti-inflammatory agent and anti-oxidant agents, examples of which are provided in greater detail below. The skin anti-inflammatory compound may be added to the antiperspirant and deodorant composition in an amount between about 1-20% by weight, about 1-10% by weight, or about 1-5% by weight of the total antiperspirant composition. (Please make the minimum about 0.10%)

Regarding anti-inflammatory active compounds, all anti-inflammatory active ingredients or active ingredients relieving reddening and/or itching which are suitable for or commonly used for cosmetic and/or dermatological applications can be used here. Steroidal anti-inflammatory substances of the corticosteroid type, such as e.g. hydrocortisone, dexamethasone, dexamethasone phosphate, methyl prednisolone or cortisone, or other steroidal anti-inflammatories are advantageously used as anti-inflammatory active ingredients or active ingredients relieving reddening and/or itching. Non-steroidal anti-inflammatories can also be used, such as for example oxicams such as piroxicam or tenoxicam; salicylates such as aspirin, disalcid, solprin or fendosal; acetic acid derivatives such as diclofenac, fenclofenac, indomethacin, sulindac, tolmetin or clindanac; fenamates such as mefenamic, meclofenamic, flufenamic or niflumic; propionic acid derivatives such as ibuprofen, naproxen, benoxaprofen or pyrazoles such as phenylbutazone, oxyphenylbutazone, febrazone or azapropazone. Alternatively, natural anti-inflammatory substances or substances to relieve reddening and/or itching can be used. Plant extracts, special highly active plant extract fractions and highly pure active substances isolated from plant extracts can be used. Particularly preferred are extracts, fractions and active substances from camomile, aloe vera, commiphora species, rubia species, willow, willowherb, oats, calendula, arnica, St. John's wort, honeysuckle, rosemary, Passiflora incarnata, witch hazel as well as pure substances such as inter alia bisabolol, apigenin, apigenin-7-glucoside, rosemarinic acid, boswellic acid, phytosterols, glycyrrhizinic acid, glabridin or licochalcone A. The formulations can also contain mixtures of two or more anti-inflammatory active ingredients.

Regarding anti-oxidant compounds, all antioxidants that are suitable for or commonly used for cosmetic and/or dermatological applications can be used. The antioxidants are advantageously selected from the group consisting of amino acids (e.g., glycine, histidine, tyrosine, tryptophane) and derivatives thereof, imidazoles (e.g., urocanic acid) and derivatives thereof, peptides such as D, L-carnosine, D-camosine, L-carnosine and derivatives thereof (e.g., anserine), carotenoids, carotenes (e.g., α-carotene, β-carotene, lycopene) and derivatives thereof, chlorogenic acid and derivatives thereof, lipoic acid and derivatives thereof (e.g., dihydrolipoic acid), aurothioglucose, propyl thiouracil and other thiols (e.g., thioredoxin, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and the salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g., buthionine sulfoximines, homocysteine sulfoximine, buthionine sulfones, penta-, hexa-, heptathionine sulfoximine) in very small compatible doses, also (metal) chelators, e.g. α-hydroxy fatty acids, palmitic acid, phytic acid, lactoferrin, α-hydroxy acids (e.g., citric acid, lactic acid, malic acid), humic acid, bile acid, bile extracts, bilirubin, biliverdin, EDTA, EGTA and derivatives thereof, unsaturated fatty acids and derivatives thereof (e.g., γ-linolenic acid, linoleic acid, oleic acid), folic acid and derivatives thereof, ubiquinone and ubiquinol and derivatives thereof, vitamin C and derivatives (e.g., ascorbyl palmitate, Mg ascorbyl phosphate, ascorbyl acetate), tocopherols and derivatives thereof (e.g. vitamin E acetate), vitamin A and derivatives thereof (vitamin A palmitate) as well as coniferyl benzoate of benzoic resin, rutic acid and derivatives thereof, α-glucosyl rutin, quercetin and derivatives thereof, rosemarinic acid, carnosol, carnosolic acid, resveratrol, ferulic acid and derivatives thereof, furfurylidene glucitol, butyl hydroxytoluene, butyl hydroxyanisole, nordihydroguaiacic resin acid, nordihydroguaiaretic acid, trihydroxybutyrophenone, uric acid and derivatives thereof, mannose and derivatives thereof, superoxide dismutase, zinc and derivatives thereof (e.g., ZnO, ZnSO₄) selenium and derivatives thereof (e.g., selenium methionine), stilbenes and derivatives thereof (e.g. stilbene oxide, trans-stilbene oxide) along with derivatives (salts, esters, ethers, sugars, nucleotides, nucleosides, peptides and lipids) of these cited active ingredients or extracts or fractions of plants having an antioxidant effect, such as e.g. green tea, rooibos, honeybush, grape, rosemary, sage, melissa, thyme, lavender, olive, oats, cocoa, ginkgo, ginseng, liquorice, honeysuckle, sophora, pueraria, pinus, citrus, Phyllanthus emblica or St. John's wort.

Antiperspirant and Deodorant Formulations

As noted above, antiperspirant and deodorants including the above-noted skin lubricating compositions and anti-inflammatory compositions may be formulated broadly is any of easy-to-apply form, such as aerosols, pumps, sprays, liquids, roll-ons, lotions, creams, sticks, and soft solids, etc. Exemplary formulations for sticks and aerosols are provided herein for purposes of illustration, but are not intended to be limiting on the presently described embodiments.

In one embodiment, an antiperspirant and deodorant composition of the present disclosure may be formulated as a stick antiperspirant/deodorant. The stick antiperspirant/deodorant formulation may include an anhydrous, hydrophobic vehicle, which includes a volatile silicone and/or high melting point component, and the above-noted skin lubricating compositions and anti-inflammatory compositions suspended in the anhydrous, hydrophobic vehicle. The skin lubricating compositions and anti-inflammatory compositions are preferably in the effective amounts described above.

The high melting point components may include any material suitable for use in an antiperspirant/deodorant stick that melts at a temperature of about 70° C. or higher. Typical of such materials are the high melting point waxes. These include beeswax, spermaceti, carnauba, bayberry, candelilla, montan, ozokerite, ceresin, paraffin waxes, semi-microcrystalline and microcrystalline waxes, hydrogenated jojoba oil, and hydrogenated castor oil (castor wax). The preferred wax is hydrogenated castor oil. Other suitable high melting point components include various types of high melting point gelling agents such as polyethylene-vinyl acetate copolymers, polyethylene homopolymers, 12-hydroxystearic acid, and substituted and unsubstituted dibenzylidene alditols. Typically, the high melting point components include about 1 to about 25 wt. %, preferably about 2 to about 15 wt. %, of the composition. Volatile silicones include cyclomethicones and dimethicones, discussed above.

Other components may include, for example, non-volatile silicones, polyhydric alcohols having 3-6 carbon atoms and 2-6 hydroxy groups, fatty alcohols having from 12 to 24 carbon atoms, fatty alcohol esters, fatty acid esters, fatty amides, non-volatile paraffinic hydrocarbons, polyethylene glycols, polypropylene glycols, polyethylene and/or polypropylene glycol ethers of C₄-C₂₀ alcohols, polyethylene and/or polypropylene glycol esters of fatty acids, and mixtures thereof. The term “fatty” is intended to include hydrocarbon chains of about 8 to 30 carbon atoms, preferably about 12 to 18 carbon atoms.

Non-volatile silicones include polyalkylsiloxanes, polyalkylaryl siloxanes, and polyethersiloxanes with viscosities of about 5 to about 100,000 centistokes at 25° C., polymethylphenylsiloxanes with viscosities of about 15 to about 65 centistokes, and polyoxyalkylene ether dimethylsiloxane copolymers with viscosities of about 1200 to about 1500 centistokes.

Useful polyhydric alcohols include propylene glycol, butylenes glycol, dipropylene glycol and hexylene glycol. Fatty alcohols include stearyl alcohol, cetyl alcohol, myristyl alcohol, oleyl alcohol, and lauryl alcohol. Fatty alcohol esters include myristyl lactate, C₁₂₋₁₅ alcohols benzoate, cetyl acetate, and myristyl octanoate. Fatty acid esters include isopropyl palmitate, myristyl myristate, and glyceryl monostearate. Fatty amides include stearamide MEA, stearamide MEA-stearate, lauramide DEA, and myristamide MIPA.

Non-volatile paraffinic hydrocarbons include mineral oils and branched chain hydrocarbons with about 16 to 68, preferably about 20 to 40, carbon atoms. Suitable polyethylene glycols and polypropylene glycols will typically have molecular weights of about 500 to 6000, such as PEG-10, PEG-40, PEG-150 and PPG-20, often added as rheology modifiers to alter product appearance or sensory attributes.

Polyethylene and/or polypropylene glycol ethers or C₄-C₂₀ alcohols include PPG-10 butanediol, PPG-14 butyl ether, PPG-5-buteth-7, PPG-3-isostearth-9, PPG-3-myreth-3, oleth-10, and steareth-20. Polyethylene and/or polypropylene glycol esters of fatty acids include PEG-8 distearate, PEG-10 dioleate, and PPG-26 oleate. These are generally added to give emollient properties.

The antiperspirant/deodorant product contemplated herein also may include additives, such as those used in conventional deodorants. For example, in addition to, or instead of, antiperspirant and deodorant efficacy, the antiperspirant/deodorant product may include additives that cause the antiperspirant/deodorant product to exhibit long-lasting fragrance, odor protection, and/or another desired purpose and/or function. These additives include, but are not limited to, fragrances, including encapsulated fragrances, dyes, pigments, preservatives, moisturizers, and the like. These optional ingredients can be included in the deodorant product in an amount of about 0 to about 20 wt. %.

The above list of materials is by way of example only and is not intended to be a comprehensive list of all potential components of the antiperspirant/deodorant products contemplated herein. Other high and low melting waxes, volatile and non-volatile compounds and other suitable components are readily identifiable to those skilled in the art. Of course, other ingredients such as colloidal silica, fumed silica, particulate polyolefins, talcum materials, colorants and preservatives may also be included as desired. For example, the composition may include up to about 10% fragrance or about 2% colorant by weight.

As noted initially above, in addition to the skin lubricating compositions and anti-inflammatory compositions, the antiperspirant/deodorant formulation may include an active antiperspirant compound. The active antiperspirant compounds contain at least one active ingredient, typically metal salts, that are thought to reduce perspiration by diffusing through the sweat ducts of apocrine glands (sweat glands responsible for body odor) and hydrolyzing in the sweat ducts, where they combine with proteins to form an amorphous metal hydroxide agglomerate, plugging the sweat ducts so perspiration cannot diffuse to the skin surface. Some active antiperspirant compounds that may be used include astringent metallic salts, especially inorganic and organic salts of aluminum, zirconium, and zinc, as well as mixtures thereof. Particularly preferred are aluminum-containing and/or zirconium-containing salts or materials, such as aluminum halides, aluminum chlorohydrates, aluminum hydroxyhalides, zirconyl oxyhalides, zirconyl hydroxyhalides, and mixtures thereof. Exemplary aluminum salts include those having the general formula Al₂(OH)ₐCl_{b}x(H₂O), wherein a is from 2 to about 5; the sum of a and b is about 6; x is from about 1 to about 6; and wherein a, b, and x may have non-integer values. Exemplary zirconium salts include those having the general formula ZrO(OH)₂₋ₐClₐx(H₂O), wherein a is from about 1.5 to about 1.87, x is from about 1 to about 7, and wherein a and x may both have non-integer values. Particularly preferred zirconium salts are those complexes that additionally contain aluminum and glycine, commonly known as ZAG complexes. These ZAG complexes contain aluminum chlorohydroxide and zironyl hyroxy chloride conforming to the above-described formulas. Examples of active antiperspirant compounds suitable for use in the various embodiments contemplated herein include aluminum dichlorohydrate, aluminum-zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum-zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, aluminum sulfate buffered, and the like, and mixtures thereof. In a preferred embodiment, the antiperspirant compound is aluminum zirconium trichlorohydrex. In another embodiment, the antiperspirant product includes an active antiperspirant compound present in the amount of 0 to about 25 wt. % (USP). As used herein, weight percent (USP) or wt. % (USP) of an antiperspirant salt is calculated as anhydrous weight percent in accordance with the U.S.P. method, as is known in the art. This calculation excludes any bound water and glycerin.

The antiperspirant/deodorant composition, in an exemplary embodiment, is prepared by combining the suspending agents in the carrier. Any suitable form of mixing can be used to combine the ingredients, such as high shear mixing, stirring, agitation, blending, or any combination thereof. The active antiperspirant compound is added to the suspending agents and carrier to form a premix. Mixing continues until the premix is homogenous and fluid in consistency. The structurants are added to a mixing vessel and heat not exceeding about 85° C. is applied to melt the ingredients. As the ingredients melt, agitation is slowly commenced. The mixture, if necessary, is further cooled to about 64-69° C. and, with continuous agitation, the premix is incrementally added to the mixture until the mixture is homogenous. Additional carrier is added to the mixture with agitation such that the mixture is maintained at a temperature of about 60° C. Additives, including the anti-inflammatory and the skin lubricating compound(s), and further including compounds such as fragrances, dyes, corn starch, etc., are added with mixing while maintaining the mixture at 60° C. The final mixture is cooled to about 53° C., poured into molds, and then allowed to cool to room temperature. As used herein, the term “allowed to cool” means exposing the mixture to room temperature for a time sufficient for the mixture to come to room temperature or exposing the mixture to a refrigerator or cooling room, fan, or other cooling mechanism that lowers the temperature of the mixture to room temperature. It will be appreciated that the sequence of addition and/or combination of the various components of the antiperspirant product is not necessarily critical, and various sequences for addition or combination of the components can be used.

In another exemplary formulation, as noted above, the antiperspirant/deodorant product may be formulated as an aerosol. In this embodiment, the antiperspirant/deodorant composition has an anhydrous aerosol formulation, which is preferably substantially free of water and is stored in a container. The aerosol formulation includes the anti-inflammatory compositions, the skin lubricating compositions, and includes other various ingredients as discussed in the foregoing paragraphs, such as, for example, an active antiperspirant compound, talc, a volatile silicone, and one or more fragrances, and further includes a gaseous propellant, such as, one or more types of light hydrocarbons and/or fluorocarbons. The antiperspirant/deodorant product is contained in the container under pressure from the gaseous propellant. With regard to the container under pressure, when a plunger cap is depressed, a valve stem is actuated against a spring to an open position allowing the liquid antiperspirant/deodorant product to be forced, via a gaseous propellant, through a tube, the valve stem and finally out of a small hole in the plunger cap as an aerosol or mist. Other suitable aerosol valve-container arrangements known to those skilled in the art may also be used with the various embodiments of the present disclosure.

While two exemplary antiperspirant/deodorant formulation embodiments have been provided in detail, it will be appreciated that the antiperspirant/deodorant product may be formulated, using the above-described anti-inflammatory and skin lubricating compositions (and other option ingredients as described above, such as active antiperspirants), according to any one of aerosols, pumps, sprays, liquids, roll-ons, lotions, creams, gels, sticks, and soft solids, among others. The skilled artisan will readily be able to provide such formulations based on the present disclosure and the level of ordinary skill in the art.

### Methods of Use

As initially noted above, embodiments of the present disclosure are suitable for use as a single product to eliminate the need to use a separate antiperspirant/deodorant, lubricated razor, and an anti-inflammatory product. Thus, the user may apply the described product to the underarm (axilla) region, and then perform routine underarm shaving at any point after application and prior to or concurrent with the subsequent cleansing. The razor used in shaving need not include a skin lubricating strip, as a skin lubricating agent has been provided in the described antiperspirant/deodorant product. Further, the user need not apply a separate skin calming/anti-inflammatory agent after shaving is complete, as an anti-inflammatory agent has been provided in the described antiperspirant/deodorant product. Thus, the described embodiments are intended to provided an “all-in-one” product intended to aid the user in reducing the number of separate products applied to the underarm during the user's typical personal ear care routine.

Accordingly, the present disclosure has provided antiperspirant and deodorant compositions that effectively reduce the incidence of underarm damage during shaving of the underarm region. Additionally, the present disclosure has provided antiperspirant and deodorant compositions that reduce inflammation that may be caused by shaving the underarm region. The described deodorant products have been provided in an easy-to-apply form, such as aerosols, pumps, sprays, liquids, roll-ons, lotions, creams, gels, sticks, and soft solids, etc.

While at least one exemplary embodiment has been presented in the foregoing detailed description of the disclosure, it should be appreciated that a vast number of variations exist. It should also be appreciated that the exemplary embodiment or exemplary embodiments are only examples, and are not intended to limit the scope, applicability, or configuration of the disclosure in any way. Rather, the foregoing detailed description will provide those skilled in the art with a convenient road map for implementing an exemplary embodiment of the disclosure, it being understood that various changes may be made in the function and arrangement of elements described in an exemplary embodiment without departing from the scope of the disclosure as set forth in the appended claims and their legal equivalents.

What is claimed is:

1. An antiperspirant composition comprising:
an antiperspirant active compound based upon an astringent metallic salt;
a skin lubricating agent that reduces a coefficient of friction between skin and a razor blade applied to the skin; and
an anti-inflammatory agent that reduces effects of skin irritation and damage.

2. The antiperspirant composition of claim 1, wherein the antiperspirant active compound is selected from the group consisting of: salts of aluminum, zirconium, zinc, and mixtures thereof.

3. The antiperspirant composition of claim 2, wherein the antiperspirant active compound comprises a salt of aluminum and zirconium having a ratio of aluminum to zirconium of greater than about 6.0:1.0.

4. The antiperspirant composition of claim 1 further comprising one or more of fragrances, dyes, pigments, emollients, and preservatives.

5. The antiperspirant composition of claim 1, wherein the composition is provided in a stick form.

6. The antiperspirant composition of claim 5, further comprising a structurant.

7. The antiperspirant composition of claim 1, wherein the composition is provided in an aerosol form.

8. The antiperspirant composition of claim 7, further comprising a gaseous propellant.

9. The antiperspirant composition of claim 1, wherein the skin lubricating agent comprises a quaternary ammonium compound.

10. The antiperspirant composition of claim 1, wherein the skin lubricating agent comprises a synthetic polymer.

11. The antiperspirant composition of claim 1, wherein the skin lubricating agent comprises a fatty ester.

12. The antiperspirant composition of claim 1, wherein the anti-inflammatory agent comprises a non-steroidal anti-inflammatory compound.

13. The antiperspirant composition of claim 1, wherein the anti-inflammatory agent comprises a plant extract.

14. The antiperspirant composition of claim 1, wherein the anti-inflammatory agent comprises an anti-oxidant.

15. The antiperspirant composition of claim 14, wherein the anti-oxidant comprises a tocopherol compound or a tocopheryl derivative.

16. The antiperspirant composition of claim 14, wherein the anti-oxidant comprises an ascorbic acid compound or an ascorbate derivative.

17. The antiperspirant composition of claim 1, wherein the skin lubricating agent is present in an amount of about 1-5% by weight of the total antiperspirant composition.

18. The antiperspirant composition of claim 1, wherein the anti-inflammatory agent is present in an amount of about 0.10%-5% by weight of the total antiperspirant composition.

19. A method of using an antiperspirant composition comprising:
applying to the underarm region an antiperspirant composition comprising an antiperspirant active compound based upon an astringent metallic salt, a skin lubricating agent that reduces a coefficient of friction between skin and a razor blade applied to the skin, and an anti-inflammatory agent that reduces effects of skin irritation and damage; and
subsequent to applying the antiperspirant composition, shaving the underarm region using a razor.

20. The method of claim 19, wherein shaving comprises shaving with a razor that does not include a lubricating strip, and wherein the method excludes applying an anti-inflammatory product to the underarm region immediately subsequent to shaving the underarm region.
